(19) United States
(12) Patent Application Publication
(10) Pub. No.: US 2025/0065117 A1
(43) Pub. Date: Feb. 27, 2025

(54) TONIC MOTOR ACTIVATION TO IMPROVE PATIENT SLEEP

(71) Applicant: Noctrix Health, Inc., Pleasanton, CA (US)

(72) Inventors: Jonathan David Charlesworth, San Francisco, CA (US); Shriram Raghunathan, Castro Valley, CA (US); Jatin Murlidhar Tekchandani, Humble, TX (US); Stephanie Kayla Rigot, Pleasanton, CA (US)

(21) Appl. No.: 18/811,165

(22) Filed: Aug. 21, 2024

Related U.S. Application Data
(60) Provisional application No. 63/533,786, filed on Aug. 21, 2023.

(51) Int. Cl. A61N 1/36

(52) U.S. Cl. CPC A61N 1/36003; A61N 1/36031; A61N 1/36034

(57) ABSTRACT

A method for treating a patient via high-frequency (HF) electrostimulation therapy at an external target body location comprises generating a tonic motor activation (TOMAC) electrostimulation waveform using a power source supplying less than 100 volts zero-to-peak voltage amplitude per phase. The waveform has a frequency of 1,000 Hz to 5,000 Hz, a zero-to-peak current amplitude of 10-50 mA, and a duty cycle greater than 25%. The waveform is configured to overcome skin capacitance, induce proprioceptive afferent activity, and induce sustained tonic motor activation of a muscle. The method involves delivering the waveform to the external target body location over a specified duration during the patient's sleep period and receiving at least one TOMAC efficacy indication. The efficacy indication may relate to sleep efficiency, sleep stage, sleep onset latency, daytime sleepiness, CPAP compliance, or patient physiological response indicating TOMAC.

### CLAIM OF PRIORITY

This application claims priority to U.S. Provisional Application Ser. No. 63/533,786, filed on Aug. 21, 2023, which is incorporated by reference herein in its entirety, and the benefit of priority of which is claimed herein.

### BACKGROUND

The field of electrostimulation therapy can encompass a wide range of applications, from pain management to neuromuscular rehabilitation. High-frequency electrostimulation has shown promise in various therapeutic contexts due to its ability to interact with the human nervous system in unique ways. Electrical nerve stimulation can be used to treat one or more conditions, such as chronic or acute pain, epilepsy, depression, bladder disorders, or inflammatory disorders. Certain neurological disorders can be attributed to overactivity of sensory or other peripheral nerve fibers which can disrupt quality of life, and/or the processing of such neural activity in the brain.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various embodiments discussed in the present document.

FIG. 1A depicts respiratory events during REM sleep as compared to other non-REM sleep states.

FIG. 1B depicts respiratory events during REM sleep as compared to other non-REM sleep states.

FIG. 1C depicts hypopnea and obstructive apnea event frequencies during REM sleep as compared to other non-REM sleep states.

FIG. 1D depicts hypopnea and obstructive apnea event frequencies during REM sleep as compared to other non-REM sleep states.

FIG. 2 depicts a system for sleep monitoring including an electrostimulation device for effecting tonic motor activation for reducing rapid eye movement sleep.

FIG. 3A is a chart showing polysomnography (PSG) data of tonic motor activation (TOMAC).

FIG. 3B is a chart showing PSG data of tonic motor activation (TOMAC).

FIG. 3C is a chart showing PSG data of tonic motor activation (TOMAC).

FIG. 3D is a chart showing PSG data of tonic motor activation (TOMAC).

FIG. 3E is a chart showing PSG data of tonic motor activation (TOMAC).

FIG. 4 is a chart depicting a restoration of sleep during mid-night electrostimulation therapy.

FIG. 5 depicts a perspective view of a wearable electrostimulation device.

FIG. 6 is a flowchart showing a method for treating a patient via high-frequency (HF) electrostimulation therapy at an external target body location.

FIG. 7 illustrates generally an example of a block diagram of a machine.

### DETAILED DESCRIPTION

Certain sleep therapies can involve interventions that address sleep disorders and improve overall sleep quality. Such interventions can aim to influence sleep architecture, including factors such as sleep efficiency, sleep stages, and sleep onset latency.

One sleep disorder is obstructive sleep apnea (OSA), which involves repeated episodes of partial or complete upper airway obstruction during sleep, leading to intermittent hypoxia, sleep fragmentation, and increased sympathetic nervous system activity. OSA can be associated with excessive daytime sleepiness, irritability, poor concentration, impaired memory, other co-morbidities, and a decreased quality of life. The severity of OSA can be quantified using the apnea-hypopnea index (AHI), which measures the number of apneas and hypopneas per hour of sleep. One approach to CPAP therapy can involve the delivery of pressurized air through a mask to maintain airway patency during sleep. While CPAP is effective when used consistently, patient compliance can be a significant challenge. Monitoring CPAP compliance is helpful for assessing treatment efficacy and often involves tracking mask usage time and patient positional data.

Certain sleep disorders can be influenced by an amount of a certain “stage” or “state” of patient sleep. One such stage or state is Rapid Eye Movement (REM) sleep. REM sleep can be characterized by darting eye movements, loss of muscle tone, and vivid dreaming. While REM sleep can be important for memory consolidation and other cognitive functions, certain sleep disorders and conditions (e.g., OSA) can involve abnormal or excessive amounts of REM sleep. Other disorders, such as depression can be related to sleep disorders, e.g., with affected individuals experiencing relatively long REM durations and greater wake time(s) after sleep onset.

The present inventors have recognized the benefit of electrostimulation techniques to help mitigate symptoms of one or more sleep conditions, parasomnias, or certain comorbidities of a sleep disorder. Electrostimulation techniques described herein can induce tonic motor activation (TOMAC) of a patient muscle. For example, an electrostimulation waveform can be applied transdermally to induce TOMAC, e.g., to help mitigate symptoms of restless legs syndrome (RLS), periodic limb movement disorder (PLMD). The TOMAC can alternatively or additionally be induced to mitigate at least one of depression or a REM-associated parasomnia, e.g., REM behavior disorder, sleep paralysis, sleep-related painful erections, REM sleep-related sinus arrest, or nightmare syndrome. Further, the TOMAC can alternatively or additionally be induced to improve sleep efficiency, control or improve a sleep stage, reduce sleep onset latency, or reduce daytime sleepiness, improve CPAP compliance.

This document describes a technique for treating patients using high-frequency (HF) electrostimulation therapy at an external target body location. For example, a TOMAC electrostimulation waveform can be generated using a power source that supplies less than 100 volts zero-to-peak voltage amplitude per phase. This waveform can dispense charge onto patient skin with sufficient waveform parameters (e.g., frequency, current, etc.) to overcome the natural capacitance of human skin and to induce proprioceptive afferent activity of the patient. For example, parameters of the waveform can be established or adjusted to induce sustained TOMAC of a patient's muscle. In an example, the waveform can include parameters such as a frequency range of about 1,000 Hz to about 5,000 Hz, a zero-to-peak current amplitude of about 10 milliamperes (mA) to about 50 mA, and a duty cycle greater than about 25%.

The TOMAC electrostimulation waveform can be delivered to the external target body location over a specified duration, such as at least partially during the patient's sleep period. In an example, a TOMAC efficacy indicator can be received to assess or monitor treatment effectiveness. The TOMAC efficacy indicator can, include, e.g., sleep efficiency, sleep stages metric (e.g., deep sleep, rapid eye movement (REM) sleep), sleep onset latency, daytime sleepiness, continuous positive airway pressure (CPAP) compliance, a patient physiological response (e.g., a non-restless legs syndrome (RLS) and non-periodic limb movement disorder (PMLD) patient physiological response), or a combination thereof. Such efficacy indicators allow for real-time assessment and potential adjustment of the treatment parameters to optimize outcomes for each patient. In an example, the patient physiological response can include muscle activation patterns (e.g., sustained tonic activation of specific muscles, particularly the tibialis anterior muscle) measured using surface electromyography (sEMG) or polysomnographic data. Sleep architecture changes, including improvements in sleep efficiency, increased deep sleep duration, and alterations in rapid eye movement (REM) sleep patterns, can also be assessed or monitored (e.g., through polysomnography). Other indicators include reduced sleep onset latency, decreased sleep fragmentation, sustained sleep following TOMAC electrostimulation, changes in respiratory parameters such as AHI, and patient-reported outcomes like reduced daytime sleepiness and improved subjective sleep quality. The efficacy of the TOMAC electrostimulation can alternatively or additionally be evaluated through various metrics, such as reduced sleep onset latency, reduced sleep fragmentation, restored sleep during treatment, rapid sleep onset (e.g., less than ten minutes following treatment commencement), sustained sleep following the specified duration of treatment, or a combination thereof.

In an example, the TOMAC electrostimulation can be adjusted during the specified duration to reduce or inhibit obstructive sleep apnea (OSA), rapid-eye-movement (REM) sleep, REM-associated parasomnias, and depression. In an example, an inertial measurement unit (IMU) sensor can be included or used such as to track patient positional information. Here, patient positional information can be used to assess CPAP mask compliance, e.g., providing insight into the overall effectiveness of an OSA or other treatment regimen.

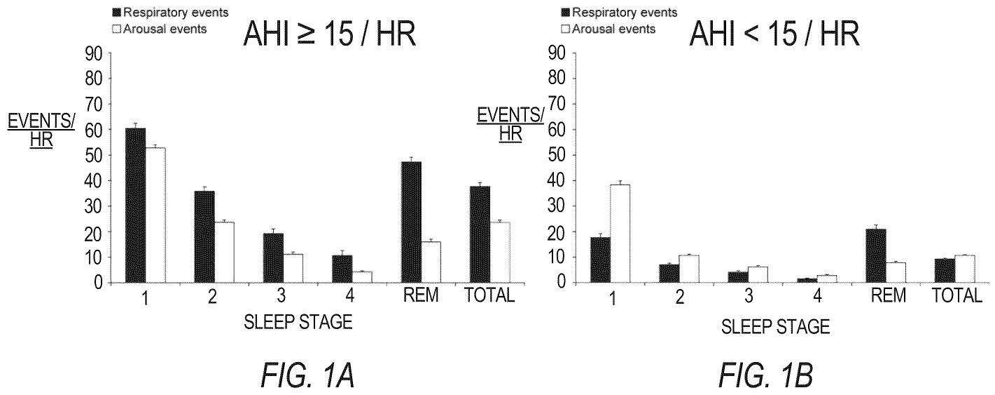

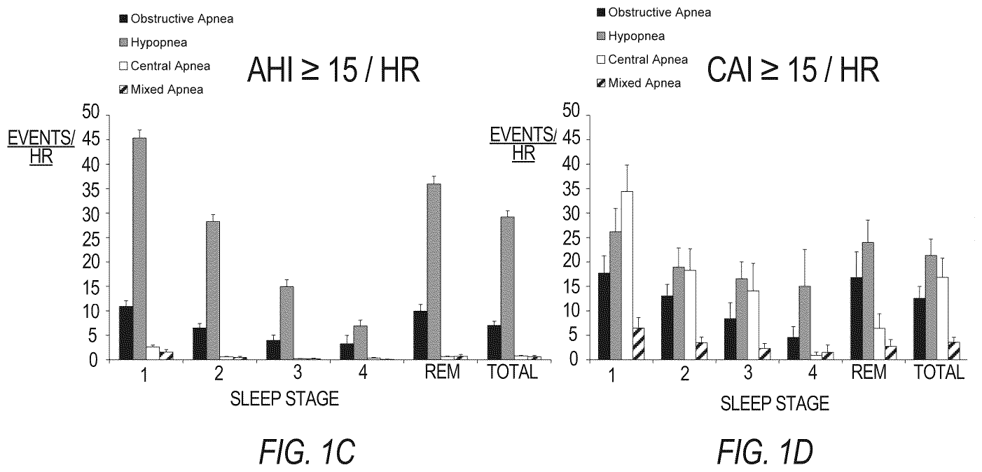

FIG. 1A and FIG. 1B show respiratory events during REM sleep as compared to other non-REM sleep states. FIG. 1C and FIG. 1D show hypopnea and obstructive apnea event frequencies during REM sleep as compared to other non-REM sleep states. As shown in FIG. 1A and FIG. 1B, individuals tend to experience more respiratory events (e.g., apneas, hypopneas, and oxygen desaturation episodes) during REM sleep than during other non-REM sleep states. Without being bound by theory, OSA may be linked to the loss of muscle tone that occurs during REM sleep, which can affect the throat and neck muscles that maintain airway patency. OSA severity can be indicated by determining an apnea-hypopnea index (AHI). For individuals with a relatively high (e.g., ≥15 events per hour) apnea-hypopnea index (AHI), such frequent respiratory events during REM sleep can have especially negative effects. REM-linked OSA can lead to chronic sleep deprivation, hypoxemia, hypertension, cardiovascular disease, stroke, and other comorbidities associated with OSA. Also, as shown in FIG. 1C and FIG. 1D, hypopnea and obstructive apnea event frequencies can be relatively high during REM sleep for individuals with a relatively high AHI and a relatively high (e.g., ≥5 events per hour) Central Apnea Index (CAI). For example, CAI can be an indicator for OSA, such as for patients with treatment-emergent central sleep apnea or other combinations of obstructive and central sleep apneas.

REM sleep abnormalities can also be associated with REM-associated parasomnias. These can include REM sleep behavior disorder (RBD), isolated recurrent sleep paralysis, nightmare disorder, and related conditions that manifest during REM sleep due to REM-specific physiology. For example, RBD involves abnormal dream-enacting behaviors during REM sleep that can result in injury to oneself or to a bed partner due to the lack of a normal degree of REM atonia. Isolated sleep paralysis also involves unwanted characteristics REM sleep during periods of wakefulness. Therefore, mitigating or reducing REM sleep may help treat the underlying pathology in these REM-associated disorders.

One approach to reducing REM sleep abnormalities can involve transcutaneous electrical nerve stimulation (TENS) techniques to modulate REM sleep alternatively or additionally to administering drugs. Here, a TENS waveform, generally within the range of 1 Hertz (Hz) to 300 Hz can be applied at a relatively low duty cycle (e.g., less than about 1:10 or less than about 1:20 ratio of a total “on” time to a total “off” time). However, it can be challenging to determine a suitable or optimal TENS waveform for a particular sleep disorder or condition such as to reduce or mitigate the REM sleep abnormality without adversely affecting other sleep states. Refining, optimizing, or validating TENS waveforms to reduce or mitigate REM sleep abnormalities, e.g., for OSA and REM-associated parasomnias, can also be challenging. The present inventors have recognized a need for improved systems and methods for treating sleep disorders and conditions involving REM sleep abnormalities.

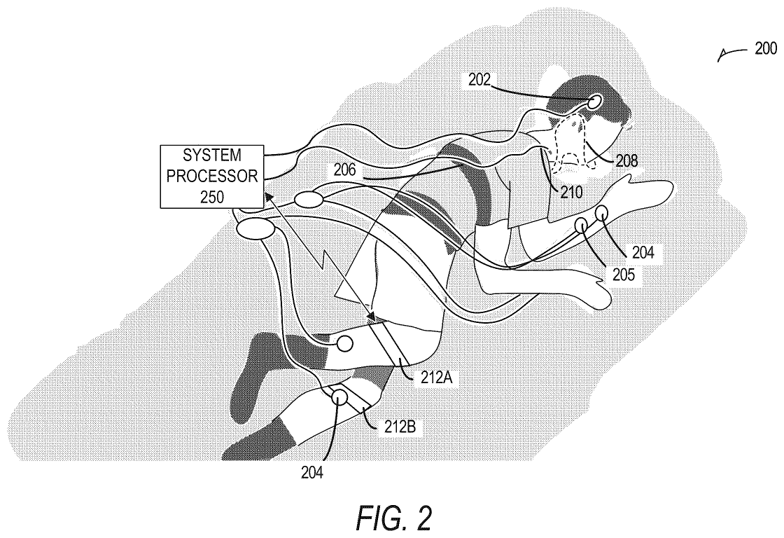

FIG. 2 depicts a system for sleep monitoring (e.g., performing polysomnography (PSG)) including an electrostimulation device for effecting tonic motor activation for reducing rapid eye movement sleep. A system 200 can include one or more of the following: head electrodes 202 arranged to monitor patient brain activity, body electrodes 204 arranged to monitor patient muscle movements, a respiration-monitoring belt 206 arranged to monitor breathing at or near a patient abdomen, a nasal cannula 208 arranged to monitor patient airflow at or near the nose, a microphone 210 configured to monitor breathing, e.g., snoring or breathing rhythm at or near the mouth, one or more wearable electrostimulation devices 212, and a system processor 250. The system 200 can also include one or more other auxiliary sensors 205, such as for measuring body temperature, heart rate, eye movement, blood oxygenation, etc. during sleep.

The system 200 can include or use the one or more wearable electrostimulation devices 212. For example, two wearable electrostimulation devices 212a and 212b can be included, such as for bilateral electrostimulation. An individual wearable electrostimulation device 212 can include an electrostimulation electronics unit and one or more electrostimulation electrodes. The wearable electrostimulation device 212 can be worn by the subject and can include or use the electrostimulation electronics unit coupled to the electrodes, such as for transcutaneously delivering an electrostimulation signal. Herein, the term electrostimulation can refer to certain high-frequency (HF) (greater than 400 Hertz, such as greater than 1 kilohertz) electrostimulation waveforms capable of inducing tonic motor activation (TOMAC) when applied at an external target body location of the patient. The wearable electrostimulation device 212 can be sized and shaped to be able to be attached or held to a body location of the subject, e.g., a leg, arm, foot, waist, neck, head, or chest of the subject. For example, the wearable electrostimulation device 212 can include or use a strap to help hold the electrodes 106 to the skin of the subject. While electrodes are generally described herein with a focus on providing electrostimulation to a subject, the electrodes can alternatively or additionally be used such as to help detect or measure one or more biosignals or biopotentials from the subject.

An individual electrostimulation device 212 can supply an alternating current (AC) electrostimulation signal for delivery to the skin at an electrode-skin interface. The electrostimulation signal can be supplied at a frequency between about 1 kHz to about 10 kHz such as for treating Restless Leg Syndrome (RLS) or Periodic Limb Movement Disorder (PLMD), such as described in Charlesworth U.S. Pat. No. 11,103,591, which is hereby incorporated by reference herein. The wearable electrostimulation device 212 can include or use a strap, sleeve, clamp, or band to help hold the electrodes to the skin of the subject. In another example, the electrostimulation device can include or use an adhesive or can connect to other items wearable by the subject, e.g., hats, clothing, etc. Alternatively, the wearable electrostimulation device 212 can be sufficiently wearable on the skin surface of the subject by adhesion forces of the electrodes alone without the need for additional features to help hold the device to the subject. The wearable electrostimulation device 212 can be attached or held to a body location of the subject, e.g., a leg, arm, foot, waist, neck, head, or chest of the subject at or near a nerve location (e.g., at or near a peroneal nerve, at or near a sural nerve, etc.) of the subject skin for transcutaneous electrostimulation thereof.

In an example, one or more electrostimulation electrodes can be configured for application at an external target body location of a subject, e.g., at or near a peroneal nerve of a leg of a subject. The electrostimulation electronics unit can generate a tonic motor activation TOMAC electrostimulation waveform for delivery, via the one or more electrodes, to the external target body location. The waveform can be generated at a frequency between 400 Hertz (Hz) and 10,000 Hz and at a specified first current between 5 milliamperes (mA) and 50 mA. For example, the TOMAC waveform can be generated at a frequency within a range of about 1 kHz (kilohertz) and 5 kHz, or about 2 kHz. The TOMAC waveform can be generated via a power source supplying less than about 100 volts zero-to-peak voltage amplitude per phase, such as less than about 60 volts zero-to-peak voltage amplitude per phase. In an example, the TOMAC waveform can be generated having a duty cycle greater than about 25%, such as a duty cycle greater than about 40%, greater than about 50%, greater than about 75%, or greater than about 90%. In an example, the TOMAC waveform can deliver a specified root means squared (RMS) current (e.g., over a specified duration such as about 10 minutes (min), about 20 min, about 30 min, about 40 min, etc.). For example, the RMS current can be between about 10 mA and about 40 mA. This approach is distinct from that of other TENS devices, which generally exhibit an RMS current between about 3 mA and about 5 mA and involve much lower frequencies (e.g., within a range of about 1 Hz to about 300 Hz) and lower duty cycles (e.g., within a range of about 2% to about 15%). The present inventors have recognized the benefits of delivering such a high frequency, high duty cycle current, including delivery of sufficient charge to induce TOMAC in the patient without discomfort or injury to the patient. Such a physiological response is not able to be induced by other electrostimulation approaches, such as TENS.

The system processor 250 can be communicatively coupled to head electrodes 202 arranged to monitor patient brain activity, body electrodes 204 arranged to monitor patient muscle movements, a respiration-monitoring belt 206 arranged to monitor breathing at or near a patient abdomen, a nasal cannula 208 arranged to monitor patient airflow at or near the nose, a microphone 210 configured to monitor breathing, e.g., snoring or breathing rhythm at or near the mouth, one or more wearable electrostimulation devices 212, or a combination thereof. For example, the system processor 250 can monitor TOMAC efficacy during the delivery of the TOMAC electrostimulation waveform via the one or more wearable electrostimulation devices 212. Here, the TOMAC efficacy can be determined based on at least one of sleep efficiency, sleep stage, sleep onset latency, daytime sleepiness, continuous positive airway pressure (CPAP) compliance, or patient physiological response (e.g., a non-RLS and non-PLMD physiological response) indicating of TOMAC. Herein, the non-RLS and non-PLMD physiological response means a physiological response not primarily or necessarily indicative of RLS or PLMD, such as a physiological response other than independent of limb movements (LMs), paresthesias or dysesthesias, etc. In an example, the TOMAC efficacy can be determined by the system processor 250 based on, e.g., sleep efficiency, sleep stages (e.g., deep sleep, rapid eye movement (REM) sleep), sleep onset latency, daytime sleepiness, continuous positive airway pressure (CPAP) compliance, a patient physiological response indicating TOMAC, or a combination thereof. For example, the patient physiological response can include muscle activation patterns (e.g., sustained tonic activation of a patient muscle, such as at the tibialis anterior muscle). In an example, the one or more auxiliary sensors 205 can include an sEMG sensor configured to measured surface electromyography (sEMG) data, and the system processor 250 can process the data to help determine the TOMAC efficacy. Alternatively or additionally, the one or more other auxiliary sensors 205 can include an inertial measurement unit (IMU) sensor, and the system processor can monitor patient positional information from the IMU. Here, the system processor 250 can receive the patient positional information to assess CPAP mask compliance, e.g., providing insight into the overall effectiveness of an OSA or other treatment regimen.

The system processor 250 can also monitor sleep architecture changes, such as improvements in sleep efficiency, increased deep sleep duration, or alterations in rapid eye movement (REM) sleep patterns (e.g., via polysomnography data from the system 200). The system processor 250 can also determine or calculate sleep onset latency, sleep fragmentation, a presence of sustained sleep following TOMAC electrostimulation, changes in respiratory parameters such as AHI. In an example, the system processor 250 can include, use, or be communicatively coupled to a user interface (UI) to receive a patient-reported outcomes such as reduced daytime sleepiness or improved subjective sleep quality. In an example, the system processor 250 can establish or adjust the TOMAC waveform to according to the determined TOMAC efficacy, such as to reduce or inhibit obstructive sleep apnea (OSA), rapid-eye-movement (REM) sleep, REM-associated parasomnias, and depression.

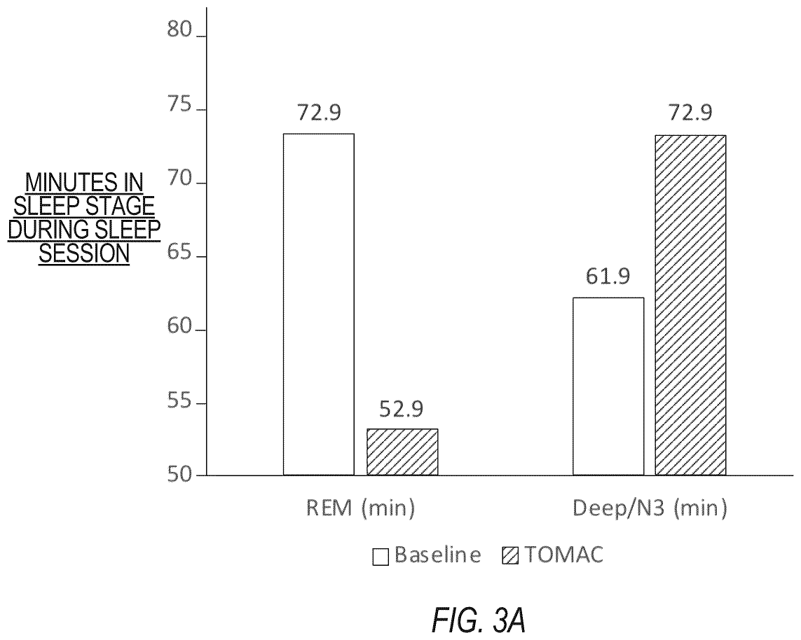

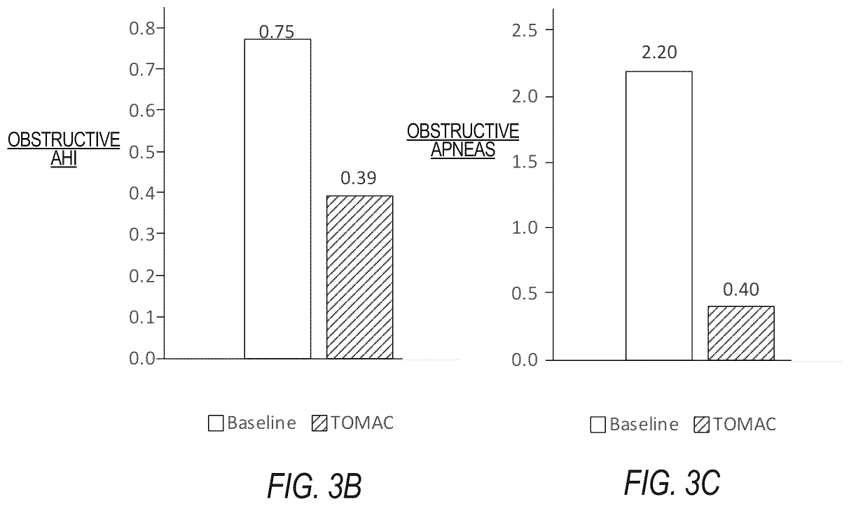

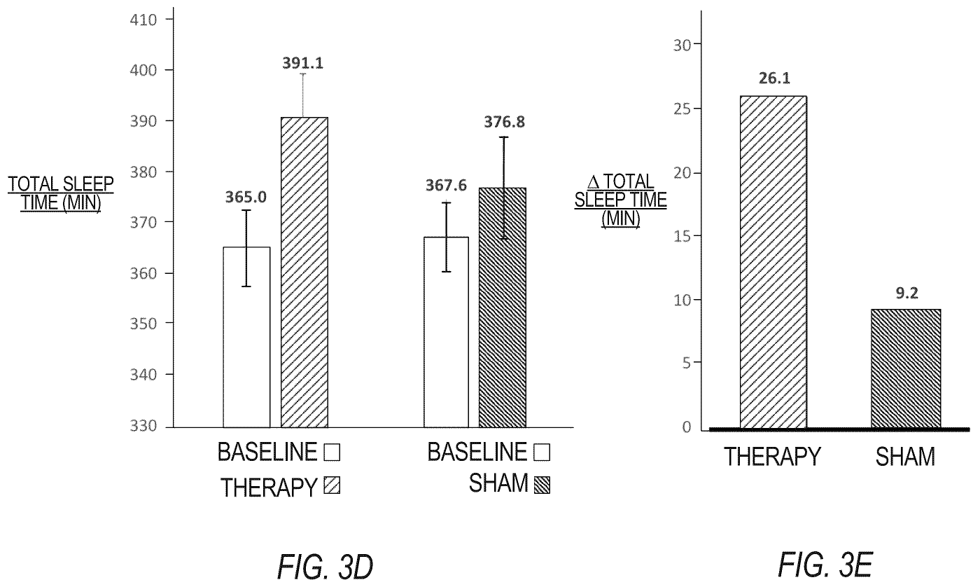

FIG. 3A, FIG. 3B, FIG. 3C, FIG. 3D, and FIG. 3E are charts each showing PSG data of tonic motor activation (TOMAC). In an example, the wearable electrostimulation device 212 (as depicted in FIG. 2) can generate an electrostimulation waveform for inducing sustained tonic motor activation (TOMAC) to the external target body location (e.g., a tibialis anterior muscle) of the subject. Herein, “sustained” TOMAC refers to the duration of the muscle response. For example, sustained TOMAC can involve a muscle response that is sustained greater than a specified duration of time (e.g., 10 seconds), and/or is sustained greater than a specified intensity threshold (e.g., about 30% of maximum muscle activation, or e.g. about 150% of minimal muscle activation for a given patient). For example, the specified intensity threshold can be patient-subject and can be quantified, e.g., as a percentage of maximum movement or force (e.g. as a result of highest voluntary contraction) by a particular patient subject.

As shown in FIG. 3A, such TOMAC can result in decrease a length of a REM sleep cycle and increase a length of a deep sleep cycle. As depicted in FIG. 3B, TOMAC can also reduce obstructive AHI by about 48% and obstructive apneas by about 80%, e.g., at relatively short durations of administering electrostimulation (e.g., between about 20 minutes and about 60 minutes, for example about 36 minutes of TOMAC per night). In another example, the wearable electrostimulation device 212 can administer the waveform such as to induce a longer TOMAC duration (e.g., between about 1 hour and about 5 hours, or between about 2 hours and about 4 hours) such as to further reduce certain symptoms or conditions of OSA.

In one example, inducing TOMAC, e.g., recurringly across several nights, can help increase a muscle tone of the subject to reduce a duration of a REM sleep state. For example, the waveform can be induced to apply TOMAC repeatedly, e.g., nightly over the course of several successive nights, and to progressively increase the duration of TOMAC each night. In another example, inducing TOMAC for an initial period can involve a persisting shift in sleep architecture, e.g., to include less REM sleep and to include more deep sleep, without a need to induce TOMAC each and every night of the entire treatment period.

In an example, a subject can be selected based on the criterion for possessing both symptoms or conditions of restless legs syndrome (RLS) and symptoms or conditions of OSA. Here, the waveform can be applied to induce TOMAC, to primarily target certain RLS symptoms, e.g., sensations of discomfort in the legs, as well as relieving an OSA symptom, such as snoring or daytime fatigue. Such a method of applying a waveform to induce TOMAC, targeting symptoms of RLS is described in Charlesworth U.S. Pat. No. 11,103,591, incorporated herein. In another example, a subject can be selected based on the criterion for possessing both symptoms of RLS and symptoms of OSA, and TOMAC can be induced for a longer duration than would be otherwise selected for targeting RLS symptoms alone. Such a longer duration can be selected, e.g., based on feedback of OSA symptoms. Here, although TOMAC is induced for a greater duration than a minimum duration needed to address RLS symptoms, the subject can receive electrostimulation therapy for both RLS and OSA without significant discomfort or disruption as compared to receiving electrostimulation therapy for RLS alone.

Similar to that described above with respect to patients having OSA symptoms, a subject can be selected based on the criterion for possessing both symptoms of restless legs syndrome (RLS) and having at least one of depression or a REM-associated parasomnia. Here, the waveform can be applied to induce TOMAC, to primarily target certain RLS symptoms, e.g., sensations of discomfort in the legs, as well as relieving at least one of depression or a REM-associated parasomnia, e.g., REM behavior disorder, sleep paralysis, sleep-related painful erections, REM sleep-related sinus arrest, or nightmare syndrome. In another example, a subject can be selected based on the criterion for possessing both symptoms or conditions of RLS and REM-associated parasomnias or depression, and TOMAC can be induced for a longer duration than would be otherwise selected for targeting RLS symptoms alone. Such a longer duration can be selected, e.g., based on feedback of depression symptoms, e.g., improvements in depression symptoms, or based on feedback of the REM-associated parasomnia symptoms, e.g., reductions in the frequency or severity of the REM-associated parasomnia. Here, although TOMAC is induced for a greater duration than a minimum duration needed to address RLS symptoms, the subject can receive electrostimulation therapy for both RLS and at least one of depression or a REM-associated parasomnia without significant discomfort or disruption as compared to receiving electrostimulation therapy for RLS alone.

As described above, at least one parameter (e.g., a duration for inducing TOMAC) of the waveform can be established or adjusted based on feedback corresponding with OSA symptoms, depression symptoms, or REM-associated parasomnia symptoms. In an example, the wearable electrostimulation device 212 (as depicted in FIG. 2) can receive data received from one or more sensors for determining an indication of an OSA symptom (e.g., to determine an AHI) a depression symptom, or a REM-associated parasomnia symptom. For example, the wearable electrostimulation device 212 can include at least one optical sensor for estimating blood oxygenation. In an example, the one or more sensors can be included in the wearable electrostimulation device 212, such as described in Charlesworth U.S. Patent Pub. No. US2023-0149711A1, which is hereby incorporated by reference herein. Alternatively or additionally, the one or more sensors can be located remote from the wearable electrostimulation device, such as including any one of the head electrodes 202, the body electrodes 204, the belt 206, the nasal cannula 208, or the microphone 210 as depicted in FIG. 2, or a remotely located continuous positive airway pressure (CPAP) machine. In an example, the one or more sensors can include an accelerometer, an inertial measurement unit (IMU), a gyroscope, an electromyography (EMG) sensor, an electroencephalography (EEG) sensor, a heart rate sensor, an oxygen sensor, or another type of sensor for sensing at least one indication of obstructive sleep apnea (OSA), rapid-eye-movement (REM) sleep, a REM-associated parasomnia, or depression.

For example, the one or more sensors can include a surface electromyographic (sEMG) sensing electrode or an array of sEMG electrodes, and individual ones of a plurality of the electrodes in the array can be selected such as to pinpoint inspection at the external target body location of the subject. Data received from an sEMG electrode can indicate the presence of muscle contractions of the subject at the external body location of the sEMG electrode that can be indicative of, e.g., an OSA event, a REM-associated parasomnia event, or a level of muscle activity that may be indicative of a depression symptom of the patient. Here, the wearable electrostimulation device (e.g., an electrostimulation electronics unit thereof) can accordingly establish or adjust one or more parameters of the TOMAC waveform based on such indications, e.g., to establish or adjust via the induced TOMAC an amount or duration of REM sleep in relation to non-REM sleep of the subject.

The one or more sensors can also include an oxygen sensor or a heart signal sensor for receiving feedback corresponding with OSA symptoms, depression symptoms, or REM-associated parasomnia symptoms. In an example, the oxygen sensor can be located on the wearable electrostimulation device (e.g., band or adhesive patch), such as to help enable collecting sensor data at external target body location of the subject. Alternatively or additionally, the oxygen sensor can be located remote from the wearable electrostimulation device, e.g., located on a separate wrist device or attachable to a finger of the subject. The oxygen sensor can include an optical sensor for detecting blood oxygenation sensor data (e.g., blood oxygenation saturation (Sp0₂) sensor data).

In a similar fashion, the heart signal sensor can be located on the wearable electrostimulation device (e.g., band or adhesive patch), such as to help enable collecting sensor data at external target body location of the subject. Alternatively or additionally, the heart signal sensor can be located remote from the wearable electrostimulation device, e.g., located at the central body portion of the user (e.g., the chest of the user). The heart rate sensor can include an optical sensor, such as a photoplethysmography (PPG) sensor, or a heart signal can be derived from a separately obtained blood pressure signal. For example, a blood pressure signal can be received via a leg-worn band of the wearable electrostimulation device carrier in a similar manner to a blood pressure cuff, and a heart rate or heart morphology signal can be thereby determined. The heart signal can alternatively or additionally be determined using an audio sensor, such as a microphone (e.g., microphone 210 of FIG. 2) or the accelerometer, which can be used to listen for heart sounds or blood pressure audio information. The heart sensor can include electrocardiogram (ECG) electrodes and sensing amplification circuitry that can be used to sense an electrical heart rate or heart morphology signal. The heart sensor can include an impedance sensor such as to sense a cardiac stroke signal by injecting a test current and measuring a response voltage—in this context, any skin-electrode impedance that is measured is simply present as a confounding component of the impedance signal from which the cardiac stroke component is to be extracted. The heart signal sensor can be used to determine one or more of heart rate, heart signal morphology, heart rate variability (HRV), or a secondary signal that can be derived from one or more of these. For example, a respiration signal can be derived from or correlated to HRV. A sleep or sleep-state indication can also be derived from or correlated to HRV. Here, the wearable electrostimulation device (e.g., an electrostimulation electronics unit thereof) can accordingly establish or adjust one or more parameters of the waveform based on data received from the oxygen sensor or the heart signal sensor, e.g., to establish or adjust via the induced TOMAC an amount or duration of REM sleep in relation to non-REM sleep of the subject.

In an example, the wearable electrostimulation device can receive data from a continuous positive airway pressure (CPAP) machine for determining an indication of OSA symptoms and such an indication can be used by the wearable electrostimulation device to establish or adjust at least one parameter of the waveform, thereby establishing or adjusting, via the induced TOMAC, an amount or duration of REM sleep in relation to non-REM sleep of the subject. CPAP machine data can include pressure levels, duration of use, and inferences drawn from such data about flow and AHI.

In an example, the external target body location can be selected such as to induce sustained TOMAC at or near genioglossus muscle, e.g., accessed transcutaneously by applying electrodes to the skin surface on the submental space (e.g., at or near the space directly below the chin) to help treat at least one symptom or condition of OSA. Here, electrostimulation electronics unit can generate a high-frequency (HF) electrostimulation waveform for delivery, via the one or more electrodes, to the genioglossus muscle. The electrostimulation waveform can be applied at or near the submental space to induce sustained tonic motor activation (TOMAC) to the genioglossus muscle. Such sustained TOMAC can involve a genioglossus muscle response that is sustained greater than about 10 seconds), sustained greater than about 30% of maximum muscle activation, or about 150% of minimal muscle activation). The electrostimulation waveform can be generated at a frequency between 1000 Hertz (Hz) and 10,000 Hz, at a specified first current between 5 milliamperes (mA) and 50 mA, and at a relatively high duty cycle (e.g., greater than about 25%, such as greater than about 50%).

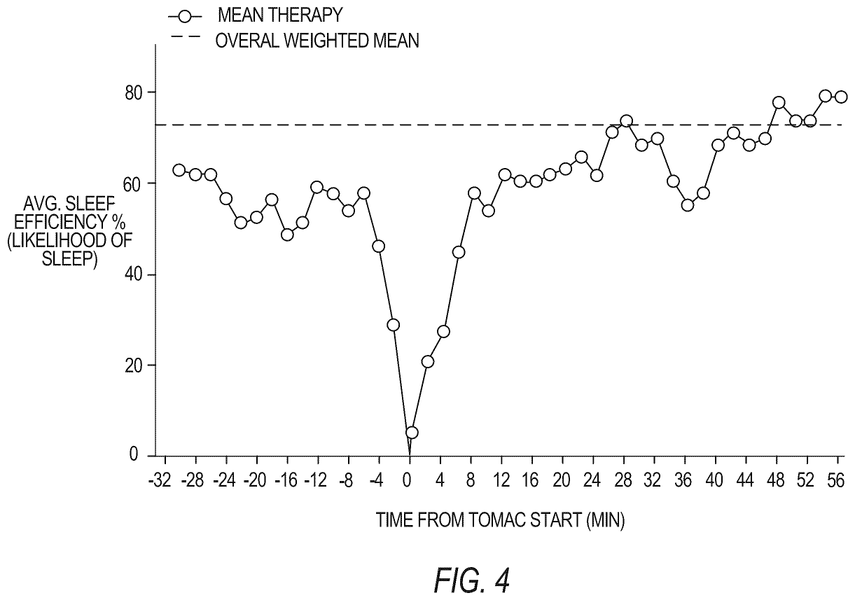

FIG. 4 is a chart depicting a restoration of sleep during mid-night electrostimulation therapy. In an example, the TOMAC efficacy indication can include an indication of at least one of reduced sleep onset latency of the patient sleep or reduced sleep fragmentation during the patient sleep. The TOMAC efficacy indication can also include an indication of restored sleep during the delivering the TOMAC electrostimulation. As shown in FIG. 4, the length of time between a patient awaking and sleep being restored following commencing delivery of the TOMAC waveform can be less than about 20 minutes, such as less than about 15 minutes, less than about 10 minutes, less than about 5 minutes, or less than about 3 minutes. In an example, regardless of whether the patient has previously been sleeping, a sleep onset can occur less than ten minutes following commencing the delivering of the TOMAC electrostimulation waveform. In an example, the TOMAC efficacy indication can include an indication that the patient remains asleep following the specified duration of delivering the TOMAC electrostimulation waveform, such as determined based in IMU data, PSG data, sEMG data, or other sensor data described herein.

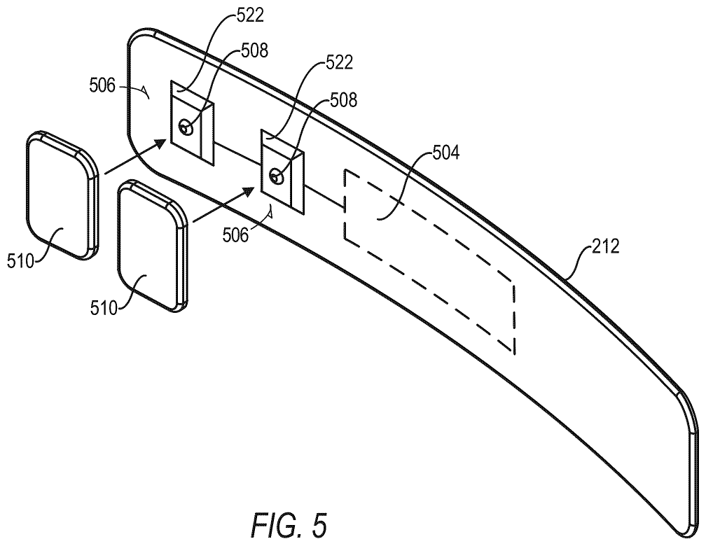

FIG. 5 depicts a perspective view of a wearable electrostimulation device. In an example, an electrostimulation therapy system can include a wearable electrostimulation device 212 including, e.g., an electrostimulation electronics unit 504, and one or more electrostimulation electrodes 506. For example, the wearable electrostimulation therapy device 212 can deliver electrostimulation therapy to skin of a subject via charge-dispersing materials such as the electrode pads 510, which help form the electrodes 506. In an example, the electrode pads 510 can be removably couplable to the wearable electrostimulation device 502. The electrode pads 510 can each be attached to a pairing surface 522 of the wearable electrostimulation device 212. The pairing surface 522 can include the electrode terminal 508 e.g., disposed therein, and the electrode terminal 508 can be electrically connected to the electrostimulation electronics unit 504. In an example, two or more electrode pads 510 are each paired to corresponding pairing surfaces 522 including one electrode terminals 508. In another example, one electrode pad 510 can be paired to an electrode pairing surface 522 containing more than one electrode terminal 508, or one electrode pad can span multiple electrode pairing surfaces 522 containing one or more electrode terminals 508. The electrode pad 510 can be removed for, e.g., hygienic maintenance, electrode maintenance such as rehydrating, or disposal.

The wearable electrostimulation device 212 can be worn by the subject and can include or use the electrostimulation electronics unit 504 coupled to the electrodes 506, such as for transcutaneously delivering an electrostimulation signal. The wearable electrostimulation device 502 can be sized and shaped to be able to be attached or held to a body location of the subject, e.g., a leg, arm, foot, waist, neck, head, or chest of the subject. In an example, the wearable electrostimulation device 212 can include or use a strap to help hold the electrodes 506 to the skin of the subject. While electrodes are generally described herein with a focus on providing electrostimulation to a subject, the electrodes can alternatively or additionally be used such as to help detect or measure one or more biosignals or biopotentials from the subject. A particular electrode 506 can include or use an electrode terminal 508 and an electrode pad 510. For example, the electrode terminal 508 can receive a capacitively-coupled (e.g., coupled using series DC-blocking capacitors, e.g., in a charge-balanced arrangement) electrostimulation signal from the electrostimulation electronics unit 504, and can deliver a resulting electrostimulation signal to the skin of the subject, such as via the electrode pad 510. In an example, multiple electrodes, such as two electrodes 506, can be used. For a bipolar electrode example having two electrodes 506, this can a first electrode that can serve as an anode and a second electrode, such as which can serve as a cathode. Also, a plurality of electrodes 506 can be arranged to form a multi-electrode group, matrix, or array such as for one or both of sensing or for delivering the electrostimulation signal to the skin of the subject. In an example, each electrode terminal 508 can be an electrode contact fixed to the wearable electrostimulation device 212 and each corresponding electrode pad 510 can be removably couplable to the device 212. In other examples, the electrode pad can be fixed to the wearable electrostimulation device 212. Because the current density of the electrostimulation signal at the electrode terminal 508 may be larger than desired, the electrode pad 510 can include an embedded or other arrangement of electrical conductors that can help distribute the electrostimulation signal current over a larger effective surface area for delivery to the subject at the skin-electrode interface. The electrode pad can be formed of a hydrogel, a hydrophilic polymer such as polyvinyl alcohol (PVA), carbon, textiles, or other types of conductive or dielectric gels, polymers, or textiles.

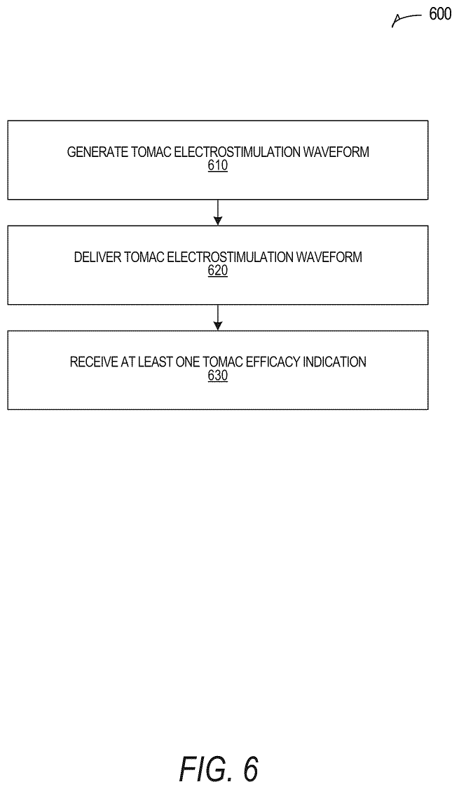

FIG. 6 is a flowchart showing a process for treating a patient via high-frequency (HF) electrostimulation therapy at an external target body location. The process 600 for a patient via high-frequency (HF) electrostimulation therapy at an external target body location can be performed using various devices and systems described herein, such as the system 200 of FIG. 2 and the electrostimulation device 212 of FIG. 2 and FIG. 5.

At 610, the tonic motor activation (TOMAC) electrostimulation waveform can be generated. For example, the TOMAC waveform can be generated via a power source supplying less than about 100 volts (e.g., less than about 60 volts) zero-to-peak voltage amplitude per phase. The TOMAC waveform can have a frequency in a range of about 1,000 Hz to about 5,000 Hz, such as at about 2,000 Hz. In an example, a zero-to-peak current amplitude of the TOMAC waveform can be established within a range of 10 milliamperes (mA) and 50 mA. In an example, a duty cycle greater than about 25%, such as greater than about 50% or greater than about 75% can be established. During the specified duration of delivering the TOMAC electrostimulation waveform, at least one parameter of the TOMAC electrostimulation can be established or adjusted to reduce or inhibit specific conditions. These conditions include obstructive sleep apnea (OSA), rapid-eye-movement (REM) sleep, REM-associated parasomnias, or depression.

At 620, the TOMAC electrostimulation waveform can be delivered transdermally to the patient skin. In an example, the TOMAC waveform can be delivered bilaterally at two different limbs of the patient. In an example, a capacitance of human skin at the external target body location can be overcome. Here, proprioceptive afferent activity in patient neurons can be induced via the TOMAC waveform.

At 630, at least one TOMAC efficacy indication can be received. The TOMAC efficacy indication can include, e.g., CPAP mask compliance, which can be determined based on patient positional information obtained from an inertial measurement unit (IMU) sensor. Alternatively or additionally, the TOMAC efficacy indication may include an indication of the apnea-hypopnea index (AHI), providing valuable into the effectiveness of the TOMAC to treat a patient with sleep-disordered breathing such as OSA. The TOMAC efficacy indication can include an indicator of sleep efficiency, sleep onset latency, sleep fragmentation, or restored sleep during the delivery of the TOMAC electrostimulation.

In an example, the process 600 includes receiving an indication of sleep onset in response to delivering the TOMAC electrostimulation waveform, where sleep onset occurs less than ten minutes following the commencement of the TOMAC electrostimulation waveform delivery. Additionally, the process 600 can include receiving an indication that the patient remains asleep following the specified duration (e.g., greater than about 30 min, about 20 min, about 15 min, about 10 min, or about 5 min) of delivering the TOMAC electrostimulation waveform, suggesting sustained sleep improvement.

In an example, the efficacy indication can be based on physiological effects or a patient-subjective response following the delivery of the TOMAC waveform. Such a physiological response can include the tonic or sustained activation of a muscle of the patient. For example, the physiological response can be measured and indicated by surface electromyography (sEMG) data or polysomnographic data, e.g., providing quantifiable evidence of the TOMAC efficacy.

In an example, the process 600 can include performing targeted patient selection. Patients can be identified for TOMAC electrostimulation therapy based on one or more prior symptoms of Restless Legs Syndrome (RLS).

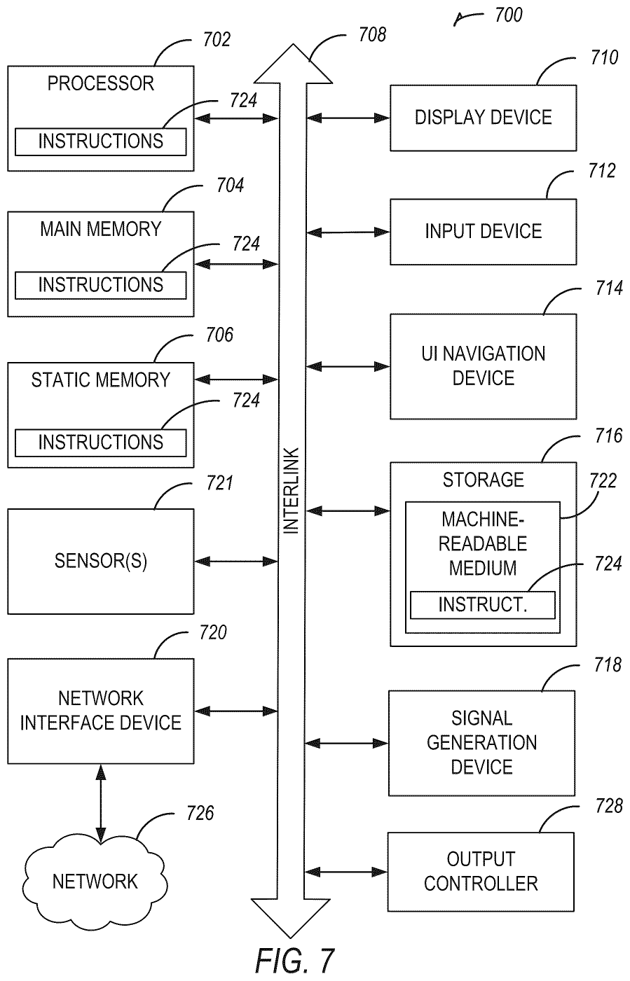

FIG. 7 illustrates generally an example of a block diagram of a machine 700 upon which any one or more of the techniques (e.g., methodologies) discussed herein may perform in accordance with some examples. In alternative embodiments, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations when operating. A module includes hardware. In an example, the hardware may be specifically configured to carry out a specific operation (e.g., hardwired). In an example, the hardware may include configurable execution units (e.g., transistors, circuits, etc.) and a computer readable medium containing instructions, where the instructions configure the execution units to carry out a specific operation when in operation. The configuring may occur under the direction of the execution units or a loading mechanism. Accordingly, the execution units are communicatively coupled to the computer readable medium when the device is operating. In this example, the execution units may be a member of more than one module. For example, under operation, the execution units may be configured by a first set of instructions to implement a first module at one point in time and reconfigured by a second set of instructions to implement a second module.

Machine (e.g., computer system) 700 may include a hardware processor 702 (e.g., a central processing unit (CPU), a graphics processing unit (GPU), a hardware processor core, or any combination thereof), a main memory 704 and a static memory 706, some or all of which may communicate with each other via an interlink (e.g., bus) 708. The machine 700 may further include a display unit 710, an alphanumeric input device 712 (e.g., a keyboard), and a user interface (UI) navigation device 714 (e.g., a mouse). In an example, the display unit 710, alphanumeric input device 712 and UI navigation device 714 may be a touch screen display. The machine 700 may additionally include a storage device (e.g., drive unit) 716, a signal generation device 718 (e.g., a speaker), a network interface device 720, and one or more sensors 721, such as a global positioning system (GPS) sensor, compass, accelerometer, or another sensor. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 716 may include a machine readable medium 722 that is non-transitory on which is stored one or more sets of data structures or instructions 724 (e.g., software) embodying or utilized by any one or more of the techniques or functions described herein. The instructions 724 may also reside, completely or at least partially, within the main memory 704, within static memory 706, or within the hardware processor 702 during execution thereof by the machine 700. In an example, one or any combination of the hardware processor 702, the main memory 704, the static memory 706, or the storage device 716 may constitute machine readable media.

While the machine readable medium 722 is illustrated as a single medium, the term “machine readable medium” may include a single medium or multiple media (e.g., a centralized or distributed database, or associated caches and servers) configured to store the one or more instructions 724.

The term “machine readable medium” may include any medium that is capable of storing, encoding, or carrying instructions for execution by the machine 700 and that cause the machine 700 to perform any one or more of the techniques of the present disclosure, or that is capable of storing, encoding or carrying data structures used by or associated with such instructions. Non-limiting machine-readable medium examples may include solid-state memories, and optical and magnetic media. Specific examples of machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 724 may further be transmitted or received over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 720 may include one or more physical jacks (e.g., Ethernet, coaxial, or phone jacks) or one or more antennas to connect to the communications network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 700, and includes digital or analog communications signals or other intangible medium to facilitate communication of such software.

The above Detailed Description can include references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” can include “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that can include elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A method for treating a patient, including for a condition other than Restless Leg Syndrome (RLS) or Periodic Limb Movement Disorder (PLMD), via high-frequency (HF) electrostimulation therapy at an external target body location, the method comprising:
generating, via a power source supplying less than 100 volts zero-to-peak voltage amplitude per phase, a tonic motor activation (TOMAC) electrostimulation waveform having a frequency in a range of 1,000 Hz to 5,000 Hz, a zero-to-peak current amplitude within a range of 10 milliamperes (mA) and 50 mA, and a duty cycle greater than 25%, the waveform configured to:
i) overcome a capacitance of human skin at the external target body location to induce proprioceptive afferent activity; and
ii) induce sustained tonic motor activation (TOMAC) of a muscle of the patient; and
delivering the TOMAC electrostimulation waveform to the external target body location over a specified duration and at least partially during a patient sleep period; and
receiving at least one non-RLS and non-PLMD TOMAC efficacy indication, indicating at least one of sleep efficiency, sleep stage, sleep onset latency, daytime sleepiness, continuous positive airway pressure (CPAP) compliance, patient physiological response indicating of TOMAC.

2. The method of claim 1, comprising establishing or adjusting at least one parameter of the TOMAC electrostimulation during the specified duration to reduce or inhibit at least one of obstructive sleep apnea (OSA), rapid-eye-movement (REM) sleep, a REM-associated parasomnia, or depression.

3. The method of claim 1, wherein the TOMAC efficacy indication includes an indication of CPAP mask compliance based on patient positional information from an inertial measurement unit (IMU) sensor.

4. The method of claim 1, wherein the TOMAC efficacy indication includes an indication of an apnea-hypopnea index (AHI).

5. The method of claim 1, comprising identifying the patient for the TOMAC electrostimulation therapy based on one or more prior patient symptoms of Restless Legs Syndrome (RLS).

6. The method of claim 1, wherein the TOMAC efficacy indication includes an indication of at least one of reduced sleep onset latency of the patient sleep or reduced sleep fragmentation during the patient sleep.

7. The method of claim 1, wherein the TOMAC efficacy indication includes an indication of restored sleep during the delivering the TOMAC electrostimulation waveform.

8. The method of claim 1, comprising receiving an indication of sleep onset in response to the delivering the TOMAC electrostimulation waveform, wherein the sleep onset occurs less than ten minutes following commencing the delivering of the TOMAC electrostimulation waveform.

9. The method of claim 1, comprising receiving an indication that the patient remains asleep following the specified duration of delivering the TOMAC electrostimulation waveform.

10. The method of claim 1, comprising receiving an indication of the TOMAC in response to the delivering of the TOMAC electrostimulation waveform.

11. The method of claim 10, wherein the physiological response indicating of TOMAC includes tonic or sustained activation of a muscle of the patient.

12. The method of claim 11, wherein the physiological response is indicated by at least one of sEMG data or polysomnographic data.

13. The method of claim 11, wherein the muscle of the patient is tibialis anterior muscle.

14. A electrostimulation system, including for a condition other than Restless Leg Syndrome (RLS) or Periodic Limb Movement Disorder (PLMD), for treating a patient via high-frequency (HF) electrostimulation therapy at an external target body location, the system comprising:
an electrostimulation electronics unit configured to generate, via a power source supplying less than 100 volts zero-to-peak voltage amplitude per phase, a tonic motor activation (TOMAC) electrostimulation waveform having a frequency in a range of 1,000 Hz to 5,000 Hz, a zero-to-peak current amplitude within a range of 10 milliamperes (mA) and 50 mA, and a duty cycle greater than 25%, the waveform configured to:
i) overcome a capacitance of human skin at the external target body location to induce proprioceptive afferent activity; and
ii) induce sustained tonic motor activation (TOMAC) of a muscle of the patient; and
electrostimulation electrodes for distributing the TOMAC electrostimulation waveform to the external target body location over a specified duration and at least partially during a patient sleep period; and
a system processor configured to monitor non-RLS and non-PLMD TOMAC efficacy during the distributing the TOMAC electrostimulation waveform, the TOMAC efficacy determined based on at least one of sleep efficiency, sleep stage, sleep onset latency, daytime sleepiness, continuous positive airway pressure (CPAP) compliance, or non-restless legs syndrome (RLS) and non-periodic limb movement disorder (PLMD) physiological response indicating of TOMAC.

15. The system of claim 14, wherein the system processor is configured to establish or adjusting at least one parameter of the TOMAC electrostimulation during the specified duration to reduce or inhibit at least one of obstructive sleep apnea (OSA), rapid-eye-movement (REM) sleep, a REM-associated parasomnia, or depression based on the monitoring of the TOMAC efficacy.

16. The system of claim 14, comprising an inertial measurement unit (IMU) sensor configured to sense patient positional information, wherein the TOMAC efficacy indication includes an indication of CPAP mask compliance based on the patient positional information.

17. The system of claim 14, wherein the TOMAC efficacy indication includes an indication of at least one of reduced sleep onset latency of the patient sleep or reduced sleep fragmentation during the patient sleep.

18. The system of claim 14, wherein the TOMAC efficacy indication includes an indication of restored sleep during the distributing the TOMAC electrostimulation waveform.

19. The system of claim 14, comprising an auxiliary sensor configured to sense the physiological response indicating of TOMAC including tonic or sustained activation of a muscle of the patient.

20. The system of claim 19, wherein the auxiliary sensor includes polysomnography (PSG) electrodes configured for monitoring polysomnographic data as the indication of the physiological response.

21. The system of claim 19, wherein the auxiliary sensor includes surface electromyography (sEMG) electrodes configured for monitoring polysomnographic data as the indication of the physiological response.
